(19) United States
(12) Patent Application Publication
(10) Pub. No.: US 2003/0211111 A1
(43) Pub. Date: Nov. 13, 2003

(54) Antibodies to biological membranes

(76) Inventor: Charles B Hensley, Redondo Beach, CA (US)

Correspondence Address: PERKINS COIE LLP, POST OFFICE BOX 1208, SEATTLE, WA, 98111-1208, US

(21) Appl. No.: 10/258,977

(22) PCT Filed: Apr. 27, 2001
(86) PCT No.: PCT/US01/13852

(51) Int. Cl. A61K 39/395; G01N 33/53; G01N 33/567; C07K 16/18; C12P 21/04

(52) U.S. Cl. 424/178.1; 435/7.2; 435/70.21; 530/387.1; 435/317.1

(57) ABSTRACT

The present invention is directed toward antibodies specific for particular intracellular membrane components and the method of using these antibodies. In one aspect of the invention, injecting isolated membrane components into mice may generate specific antibodies to particular membrane components. The injection may include a membrane component by itself or in combination with another membrane component using an immunosuppression method. In one aspect of the invention, these antibodies are used as a novel method and system for drug delivery targeted to particular intracellular membrane components. Another aspect of the invention involves the use of these antibodies for identifying particular intracellular membrane components important for cell functions.

### FIELD OF THE INVENTION

The present invention relates to the field of biological membranes.

### BACKGROUND OF THE INVENTION

Biological membranes are important cellular structures that are generally involved in containing, compartmentalizing, and regulating the transfer of metabolites and macromolecules in a living organism. Biological membranes are composed of phospholipid bilayers associated with a range of proteins, carbohydrates, glycoproteins, glycolipids and other lipid molecules such as cholesterol. Different biological membranes have also evolved with specialized functions ranging from energy conversion in mitochondria or chloroplasts, protein production in the endoplasmic reticulum and the golgi complex, to signal or impulse relay in neural membranes. Subcellular organelles and components such as the endoplasmic reticulum, golgi complex, mitochondria, lysosomes, vacuoles, peroxisomes, chloroplast, to name a few, have specialized functions due to certain distinctive characteristics of their membranes. Hence, the ability to identify and target these particular membrane components can be important to farther elucidate and to regulate the functions of these membranes or functions associated with them.

### SUMMARY OF THE INVENTION

The present invention is directed toward antibodies specific for particular intracellular membrane components and the method of using these antibodies. In one aspect of the invention, injecting isolated membrane components into mice may generate specific antibodies to particular membrane components. The injection may include a membrane component by itself or in combination with another membrane component using an immunosuppression method. In one aspect of the invention, these antibodies are used as a novel method and system for drug delivery targeted to particular intracellular membrane components. Another aspect of the invention involves the use of these antibodies for identifying particular intracellular membrane components important for cell functions.

### GENERAL DESCRIPTION OF THE INVENTION

The articles cited below are hereby incorporated by reference as if fully set forth herein.

The present invention involves antibodies specific for particular intracellular membrane components and the method of using these antibodies. In one aspect of the invention, standard fractionation, sedimentation, density-centrifugation, and/or phase-partition techniques may be used to isolate different intracellular membrane components. These membrane components include, but are not limited to, the golgi complex, endoplasmic reticulum, mitochondria, nuclear membrane, apical membrane, basal-lateral membrane, lysosomal membrane, secretory vesicles, vacuolar membrane, tubulovesicular membranes, microtubules, and brush borders fragments of intestinal epithelial cells. These components may be isolated from different cells such as hepatocytes, intestinal epithelium, lacrimal cells, cardiomyoctes, proximal tubules, etc. Afterwards, conventional polyclonal or monoclonal antibody production methods can be used to generate antibodies that recognize these particular intracellular membrane components of the cells.

In another aspect of the invention, use of an immunosuppression method may enhance the antibody specificity toward one set of membrane components over another set of membrane components. This immunosuppression method uses cyclophosphamide and is described in Carnahan, J. and Patterson, P., The Generation of Monoclonal Antibodies that Bind Preferentially to Adrenal Chromaffin Cells and the Cells of Embryonic Sympathetic Ganglia, J. of Neuroscience, 11(11): 3493-3506. See also Matthew, W. and Patterson, P., Cyclophosphamide treatment used to manipulate the immune response for the production of monoclonal antibodies, J. Immunol. Methods 100:73-82 (1987). For example, co-injecting cyclophosphamide with an endoplasmic reticulum fraction may desensitize or make the mice immunotolerant to particular antigens represented in the endoplasmic reticulum. Following such treatments, injecting the mice with a golgi complex fraction without cyclophosphamide may preferentially immunize the mice to certain antigens represented in the golgi complex but not in the endoplasmic reticulum. This may then generate antibodies that preferentially recognize antigens in the golgi complex, but not in the endoplasmic reticulum.

In one aspect of the invention, these antibodies may be used as a novel method and system for drug delivery. In a preferred embodiment of the invention, carriers of a therapeutic agent are coupled to the antibodies generated and directed to particular intracellular membrane components of the cells. These antibodies act as targeting or homing agents that specifically deliver the therapeutic agents to the particular intracellular membrane components. Examples of the carriers can include immunoliposomes wherein the antibody or part of the antibody such as the Fab portion or a single chain of the antibody, is covalently coupled to liposomes. Park J. W., et. al., Anti-HER2 Immunoliposomes for Targeted Therapy of Human Tumors, Cancer Left. 118(2):153-60 (1997). The immunoliposomes can also include two different set of antibodies; one set of the antibodies is directed toward extracellular antigens that identify specific cells and the other set is directed toward the intracellular components of the identified cells. Thus, these immunoliposomes can first target certain cells, carry the therapeutic agent into these cells, and further home in on certain intracellular components for the therapeutic agent to act upon.

Alternatively, the antibody or part of the antibody for specific intracellular components can also be directly conjugated or covalently attached to the therapeutic agent. Injections into specific tissues such as the muscles or skin deliver the drug-modified antibody locally, and the cells can take up this drug-modified antibody by endocytosis or pinocytosis. To increase the uptake of the drug-modified antibody, it may further be modified by techniques such as cationization, as described in Pardridge, W. M., et. al., Enhanced Cellular Uptake and in vivo Biodistribution of a Monoclonal Antibody Following Cationization, J. Pharm. Sci., 84(8):943-8 (1995). The drug-modified antibody may also be delivered locally to a specific tissue using an implantable pump or encapsulating it in a biodegradable slow-release capsule.

The therapeutic agent can include various different molecules ranging from enzymes, proteins, chemicals, inhibitors, radiolabels, nucleic acids, carbohydrate moieties, etc. Numerous techniques can be used to attach these molecules to the antibody, and they are listed and described in U.S. Pat. Nos. 5,194,594, 5990,286, and 6,017,514, which are incorporated herein by reference.

In addition, these antibodies for use in drug deliveries may be humanized using techniques described in U.S. Pat. No. 5,866,692 entitled process for “Producing Humanized Chimera Antibody,” which is incorporated herein by reference. Humanization of antibodies decreases the human immune response against foreign antibodies, and thus, increases the effectiveness of these antibodies in human therapy.

Another aspect of the invention involves the use of these antibodies to aid in elucidating the functions and properties of different membrane components. These antibodies may be used as diagnostic tools to detect changes in the membrane properties in response to different cellular conditions such as apoptosis, cell division, cell resting and acquiescence, or different systemic conditions of the body such as fever, infections, inflammation, or other diseases.

The following examples illustrate the production of antibodies specific for intracellular membrane components.

### Example I

This example illustrates the isolation, fractionation, and identification of different intracellular components.

Isolation and purification of these membrane components are described in the following references and are incorporated herein by reference: Bradley, M. E., et. al., Isolation and Identification of Plasma Membrane Populations, Methods in Enzymology, 228:432-448 (1994); Mircheff, A. K., Isolation of Plasma Membranes from Polar Cells and Tissues: Apical/Basolateral Separation, Purity, Function, Methods in Enzymology, 172:18-34 (1989); Mircheff, A. K. and Van Corven Emile J. J. M., Isolation of Enterocyte Membranes, Methods of Enzymology, 192:341-354 (1990); Yang, T., et. al., MHC Class II Molecules, Cathepsins, and La/SSB Proteins in Lacrimal Acinar Cell Enidomembranes, Am J Physiol. 277(5 Pt 1):C994-C1007 (1999).

Briefly, cells from lacrimal glands, parotid glands, intestinal epithelium, proximal tubular epithelium or MDCK cells may be homogenized using a Tekmar Tissumiser at low speed (Thyristor setting of 45) for 10-20 minutes. The isolation buffer preferably consists of 5% (w/v) sorbitol, 0.5 mM naEDTA, 5 mM histidine-imidazole, pH 7.5, 9 tg/ml aprotinin, and 0.2 mM PMSF as protease inhibitor. Once homogenized, the resulting homogenate may be centrifuged at 2,000 g for 10 minutes and the supernatant is saved. To increase yield, the pellet may be re-homogenized and centrifuged while pooling the resulting supernatants.

Afterwards, the pooled supernatants may be centrifuged in a sucrose or sorbitol density gradient either designed as a continuous gradient or a stepwise gradient. For example, four milliliter aliquots of the supernatants may be first mixed with six milliliters of 87.4% sorbitol and equilibrated at 4° C. for one hour. The samples may be loaded at the junction between two hyperbolic sorbitol gradients ranging between 35 and 70% sorbitol as described in Hensley C. B. et. al., Parathryroid hornmone induced translocation of Na-H antiporters in rat proximal tubules, Am. J. Physiol., 257 (Cell Physiol. 26): C637-C645 (1989). The gradient solutions may contain 0.5 mM disodium EDTA, 0.2 mM PMSF, 9 μg/hl aprotinin, and 5 mM histidine-imidazole buffer (pH 7.5), and may be centrifuged in a swinging-bucket rotor at 100,000 g for five hours. Three-milliliter fractions may then be collected from the top using a Buchler AutoDensi-Flow and may be diluted if necessary. See Zlang, Y., et. al., Rapid distribution and inhibition of renal sodium transporters during acute pressure natriuresis, Am. J. Physiol. 270(6 Part 2): F1004-14 (1996).

A subfractionation step such as phase partitioning using thin-layer apparatus, electrophoresis, or density pertubation with digitonin may firer differentiate the different membrane components. For example, the phase partitioning may utilize a two phase system consisting of 5% Dextran T-500, 3.5% polyethyleneglycol (Carbowax) 6000, 10 pm NaEDTA, and 8.3 mM imidazole, adjusted to pH 6.6. or 7.6 with HCl. Fractions of interest from the density gradient isolation may be loaded in the thin-layer apparatus' chambers and suspended in the upper phase at a protein concentration of 1.2 mg protein/ml. The thin-layer apparatus preferably includes a fraction collector ring that holds plastic culture tubes for collecting the resulting fractions.

Further characterization of the different fractions may also utilize different biochemical markers such as enzymes that tend to localize predominantly in certain membranes. Examples of these enzymes include: Na⁺, K⁺-ATPase that is principally located in basolateral membranes; sucrase and maltase that are principally located in brush border membranes; alkaline phosphatase and γ-Glutamyltransferase that are principally located in apical membranes; galactosyltransferase that is principally located in the transgolgi elements; N-acetyl-β-D-glucosaminidase that are principally located in the lysosomes; NADPH-cytochrome-c reductase that are principally located in the endoplasmic reticulum; and succinate dehydrogenase that is principally located in the mitochondria.

### Example II

This example illustrates the immunization of mice with the isolated intracellular components.

Intracellular membrane components at about 100 μg of membrane protein in 500 μl of PBS containing 25 mM EDTA and 3 mM PMSF as protease inhibitor may be injected in RBF/m or Balb/c mice. The injection schedule may be performed as follows:

day 1: intraperitional injection of one set of intracellular membrane component (set A) with cyclophosphamide at 120 mg/kg;

day 2: intraperitional injection of cyclophosphamide only at 120 mg/kg;

day 3: repeat intraperitional injection of cyclophosphamide only at 120 mg/kg;

day 22: intraperitional injection of a second set of intracellular membrane component (set B), in Complete Freund's Adjuvant;

day 40: intravenous injection of the set B in PBS through the tail of the mouse;

day 43: the mice may be sacrificed, and their spleen cells isolated and fused using 5×10⁷ HL1-653 myeloma cells and 10⁸ splenocytes according to the method of Kohler G., et. al., Continuous Cultures of Fused Cells Secreting Antibody of Predefined Specificity, Nature 256:495497 (1975); see also Taggart R T., et. al., Stable Antibody-producing Murine Hybridomnas, Science 219:1228-1230 (1983).

Alternatively, the immunization may be performed without the immunosuppression method outlined from day 1-3. In this case, the immunization may start with only one set of intracellular membrane component at day 22 to day 43.

To determine whether the hybridoma cell lines are producing antibodies, a standard dot blot may be performed. Briefly, one μl of the supernatant from each hybridoma cell line may be applied and dried onto a nitrocellulose or nylon membrane. The nitrocellulose or nylon membrane may then be blocked and incubated with 10% normal goat serum in PBS for one hour to reduce non-specific binding of the secondary antibody. Afterwards, a goat anti-mouse IgG conjugated with horseradish peroxidase may be applied for one hour at an appropriate dilution in PBS containing 2% normal goat serum. The membranes may be washed two to three times with PBS containing 2% normal goat serum. To reveal the binding of the secondary antibody to antibodies produced by the hybridoma cell lines, the peroxidase may be incubated with standard ECL chemiluminescence substrate available from Amersham Pharmacia Biotech, Piscataway, N.J.

### EXAMPLE III

This example illustrates the screening of antibodies that recognize the different intracellular membrane components.

Nitrocellulose or nylon membranes are cut into a number of longitudinal strips, and different intracellular membrane components isolated above are spotted or applied and dried onto each strip. Blocking may be performed using casein, bovine serum albumin, or normal goat serum in PBS. Supernatant from antibody-producing hybridoma cell lines or serum from immunized mice may or may not be diluted and applied to each strip containing the different intracellular membrane components. Washing may be performed two to three times with PBS containing 2% normal goat serum, and the rest of the reaction may be performed as described in Example II above.

Alternatively, the different intracellular membrane components may be applied to a 96 well ELISA plate coated with appropriate matrix such as poly-lysine. The supernatant may or may not be diluted and applied directly into the wells, and the rest of the reaction may be accomplished using standard ELISA techniques.

### EXAMPLE IV

This example illustrates the verification of antibody specificity using immunohistochemistry.

Epithelial cells can be cultured to 80% confluent in Titek eight well glass chamber slides coated with appropriate matrix such as poly-lysine. The cells are then washed with 1×PBS and fixed with ice cold 4% paraformaldehyde. To allow access of the antibody to the intracellular components, the cell membranes may be permeabilized with 1% NP-40 or Triton-X in Tris-HCl Buffered Saline solution pH 7.4 (TBS). The antibody to be tested may be diluted appropriately in TBS and incubated for 1-2 hours at room temperature. The cells may be washed three times with TBS with 0.1% Triton X at fifteen minutes intervals. A secondary antibody specific to mouse IgG molecule with a conjugated fluorescent molecule such as fluorescein and rhodamine can be used to detected where the primary antibody binds. After washing and mounting the slides on suitable medium, the binding of the antibody in the cells can be analyzed under a fluorescence microscope.

Alternatively, intracellular binding of the antibodies may be analyzed from whole tissue sections of tissues of interests such as the liver, myocardial tissue, intestinal epithelium, or lacrimal glands. These tissues of interests can be obtained from organ donors or biopsy samples. The tissues are cut into 1-mm cubes and fixed in 4% paraformaldehyde. The tissues may be processed using standard histology techniques and sectioned using a microtome. Antibody labeling may be achieved and analyzed similarly to the cells above.

### EXAMPLE V

This example illustrates how to verify the specificity of the antibodies using electron microscopy.

Tissues of interests as described above are processed for electron microscopy analysis using standard specimen preparation techniques. Briefly, the tissues are cut into 1-mm cubes and fixed in 4% glutaraldehyde. After washing, the tissues are dehydrated in ethanol and embedded in standard electron microscope resin such as epon. The tissues are then sectioned using an ultramicrotome.

The antibodies generated in Example II are appropriately diluted and incubated with the sectioned tissues. After washing, a secondary antibody conjugated with an enzyme such as horseradish peroxidase or an opaque particle such as colloidal gold may be used to detect the primary antibody binding using an electron microscope.

### EXAMPLE VI

Processes for conjugating antibody to liposomes are described in U.S. Pat. Nos. 5,786,214, 5,210,040, and 4,957,735, which are hereby incorporated by reference as if fully set forth herein.

The preceding examples illustrate the procedures for obtaining antibodies specific toward intracellular components of the cells. They are intended only as examples and are not intended to limit the invention to these examples. It is understood that modifying and combining the examples above does not depart from the spirit of the invention.

1. An antibody specific to an intracellular membrane component, the antibody derived from a method comprising:
isolating a particular intracellular membrane component;
injecting the particular intracellular membrane component into mice; and
harvesting the antibody produced by the mice.

2. An antibody specific to an intracellular membrane component, the antibody derived from a method comprising:
isolating particular intracellular membrane components;
injecting at least one animal with a first particular intracellular membrane component together with an immunosuppression agent; and
followed by injecting said at least one animal with a second particular intracellular membrane component; without an immunosuppression agent.

3. The antibody in claim 1 or 2 wherein the antibody recognizes an intracellular membrane component selected from a group consisting of the golgi complex, endoplasmic reticulum, mitochondria, nuclear membrane, apical membrane, basal-lateral membrane, lysosomal membrane, secretory vesicles, vacuolar membrane, tubulovesicular membranes, microtubules, and brush borders fragments of intestinal epithelial cells.

4. A drug delivery system comprising the antibody in claim 1 or 2.

5. A drug delivery system comprising the antibody in claim 3.

6. A method of targeted drug delivery comprising:
generating an antibody specific to an intracellular component of the cell,
coupling the antibody to a membrane-crossing vector containing a therapeutic agent;
delivering the therapeutic agent into the cell.

7. The method in claim 6 wherein the step generating an antibody specific to a intracellular component of the cell further comprises:
isolating an intracellular membrane component;
injecting the intracellular membrane component into at least one animal; and
harvesting the antibody produced by the at least one animal.

8. The method in claim 6 wherein the intracellular membrane component includes membranes selected from a group consisting of the golgi complex, endoplasmic reticulum, mitochondria, nuclear membrane, apical membrane, basal-lateral membrane, lysosomal membrane, secretory vesicles, vacuolar membrane, tubulovesicular membranes, microtubules, and brush borders fragments of intestinal epithelial cells.

9. The method of claim 6 wherein the step of isolating specific intracellular membrane components further comprises:
lysing eulcaryotic cells; and
fractionating the specific intracellular membrane by sedimentation, densitygradient centrifugation, phase partitioning, electrophoresis, density pertubation, or any combination thereof.

10. The method of claim 9 wherein the specific intracellular membrane isolated may be identified using biochemical markers or enzymes.

11. The method of claim 6 wherein the step of isolating specific intracellular membrane components further comprises:
lysing eulcaryotic cells; and
fractionating the specific intracellular membrane by sedimentation, density-gradient centrifuigation, and phase partitioning.

12. The method of claim 6 wherein the step of isolating specific intracellular membrane components further comprises:
lysing eulcaryotic cells; and
fractionating the specific intracellular membrane by sedimentation, density-gradient centrifuigation, and electrophoresis.

13. The method of claim 6 wherein the step of isolating specific intracellular membrane components furaer comprises:
lysing eukaryotic cells; and
fractionating the specific intracellular membrane by sedimentation, density-gradient centrifuigation, and density pertubation.

14. The method in claim 6 wherein the step injecting the specific intracellular membrane components into at least one animal further comprises:
injecting the at least one animal with a first intracellular membrane component together with an immunosuppression agent; and
followed by injecting the at least one animal with a second intracellular membrane component without an immunosuppression agent.

15. A method of determining changes in biological membranes in response to external stimuli comprising the use of the antibody in claim 1 or 2.

16. A method of determining changes in biological membranes in response to external stimuli comprising the use of the antibody in claim 3.
